(19) United States
(12) Patent Application Publication
(10) Pub. No.: US 2020/0397527 A1
(43) Pub. Date: Dec. 24, 2020

(54) PACKAGING MATERIAL, METHOD FOR PRODUCING SAID PACKAGING MATERIAL, AND USE THEREOF

(71) Applicant: Huhtamaki Flexible Packaging Germany GmbH & Co. KG, Ronsberg (DE)

(72) Inventors: Johannes OBERMAYER, Lachen (DE); Andreas GMEINDNER, Oberguenzburg (DE); Markus SCHAEFTNER, Oberguenzburg (DE); Nils WALTERS, Kempten (DE)

(21) Appl. No.: 16/966,437

(22) PCT Filed: Feb. 1, 2019
(86) PCT No.: PCT/EP2019/052489

§ 371 Date: Jul. 30, 2020

(30) Foreign Application Priority Data
Feb. 5, 2018 (DE) 10 2018 102 508.4

(51) Int. Cl. A61B 50/30; D21H 27/10; D21H 19/82; D21H 19/30; D21H 19/22; B65D 65/42

(52) U.S. Cl. CPC A61B 50/30; D21H 27/10; B65D 65/42; D21H 19/30; D21H 19/22; D21H 19/824

(57) ABSTRACT

The invention relates to a packaging material for medical purposes. The packaging material comprises a packaging base material having an outer paper surface, a release lacquer that coats an entirety of the outer paper surface, and a sealing medium that is applied on the release lacquer in a grid pattern. The invention further relates to a method for producing the packaging material and its use.

### CROSS-REFERENCE TO RELATED APPLICATIONS

This application is a U.S. National Phase application submitted under 35 U.S.C. § 371 of Patent Cooperation Treaty application serial no. PCT/EP2019/052489, filed Feb. 1, 2019, and entitled PACKAGING MATERIAL, METHOD FOR PRODUCING SAID PACKAGING MATERIAL AND USE THEREOF, which claims priority to German patent application serial no. 10 2018 102 508.4, filed Feb. 5, 2018, and entitled PACKAGING MATERIAL, METHOD FOR PRODUCING THE PACKAGING MATERIAL AND USE THEREOF.

Patent Cooperation Treaty application serial no. PCT/EP2019/052489, published as WO 2019/149882 A1, and German patent application serial no. 10 2018 102 508.4, are incorporated herein by reference.

### TECHNICAL FIELD

The invention relates to a packaging material according to claim 1, a method for producing such packaging material according to claim 10 and the use of such packaging material according to claim 11.

### BACKGROUND

Such packaging materials are usually required and used as so-called “medical papers” and are used to manufacture and/or seal, in particular medical, containers and packaging.

Such packaging materials, namely “medical papers”, are usually based on a paper-based packaging base material which is coated in such a way that it can be joined to a counterpart, namely in particular glued or sealed. It is important, on the one hand, that the packaging material, or the medical paper, can be removed cleanly from the counterpart when the packaging is opened, without the packaging base material being torn during this opening process. In addition, after opening the packaging, it must be ensured that the packaging with the medical paper cannot be closed again, so that it is clearly recognizable that a packaging once opened has already been opened.

In the prior art, such packaging materials were produced by coating the paper-based packaging base material with hot melt adhesive, which was applied either in the form of a hot melt solution in toluene or directly to the paper.

However, this method and the packaging materials produced therewith comprise considerable disadvantages. When the hot-melt adhesive is applied to the paper using a solvent-based method, a high residual solvent content remains in the packaging material and the end product therefore comprises toluene.

In addition, with such a solvent-based hot-melt application, the application weight of the hot-melt adhesive is very high and ranges from 6 g/m² to 11 g/m² and higher. This high application weight of the hot-melt adhesive leads to a very high final weight of the end product on the one hand and to an exacerbation of the residual solvent problem due to the thick hot-melt adhesive layer, since the hot-melt adhesive is a sink for the solvent toluene.

On the other hand, direct application of hot-melt adhesive bears the disadvantage that the machining speed during the manufacturing of the packaging material is low, so that the productivity of the system is reduced. Furthermore, a thick hot-melt adhesive application also leads to a bad hot-tack, i.e. a poor initial seal strength when producing and/or closing the container or packaging.

These disadvantages have an unfavorable effect on the quality of previous packaging materials used as “medical paper”.

The invention is based on the objective while omitting above disadvantages to provide a packaging material which is suitable as a “medical paper”, which can be produced easily and cheaply and, in addition which ensures both, sterilizability of a product contained in a package manufactured or sealed with the packaging material and clean detachability of the packaging material from a counterpart of the package, without the possibility of resealing the package with the packaging material. Furthermore, the objective of the invention further comprises providing the production of such packaging material as well as its use.

This objective is solved by a packaging base material according to patent claim 1, by a method for producing such a packaging material according to patent claim 10 and by the use of such a packaging material according to patent claim 11.

In particular, the objective is solved by a packaging material made of paper or with an outer paper surface, the outer paper surface the outer paper surface is coated, in particular over the entire surface, with a release lacquer and the release lacquer is coated, in particular in a pattern, preferably in a grid pattern, with a sealing medium.

An essential point of the invention is that, according to the invention, a release lacquer is applied, in particular over the entire surface, to the outside of the packaging base material consisting of paper. The release lacquer ensures that the coating of the packaging base material, which consists of release lacquer and sealing medium, can be completely detached from the packaging base material, i.e. from the paper, according to a first variant without causing a paper fibre tear. When the packaging is opened, the coating of sealing medium and release lacquer originally arranged on the packaging base material thus remains on the counterpart to which the inventive packaging material was applied, so that the inventive packaging material is quasi-delaminated and thereby destroyed when the packaging is detached from the packaging or when the packaging is opened, so that resealing of the packaging is not possible under any circumstances and is thus reliably avoided.

According to the invention, the release lacquer can be applied only in the areas to be sealed on the packaging material according to the invention or, if desired, also over the entire surface of the packaging base material.

The sealing medium can also be arranged on the release lacquer over the entire surface, but preferably with a pattern, i.e. for example in a grid pattern. The sealing medium can also be applied here limited to the areas of the packaging material to be sealed according to the invention.

According to a second inventive variant, which also ensures that the inventive packaging material is structurally changed and thereby destroyed when the packaging material is detached from the packaging or opened, so that reclosing of the packaging is no longer possible under any circumstances and is therefore safely avoided according to the invention, a part of the release lacquer remains on the packaging base material. In this case, the release lacquer is broken when the packaging is opened, for example, and remains essentially at those points on the packaging base material at which the release lacquer is not coated with sealing medium if the packaging material is intact. In this variant, the release lacquer is thus not completely separated from the packaging base material; however, the sealing medium remains entirely on the counterpart with which the sealing medium was sealed when the container or packaging was formed and/or closed.

A further essential advantage of a pattern application of the sealing medium is that the inventive packaging material retains a high gas permeability if the sealing medium is not applied over the entire surface, so that sterilizability of the packaged product with ethylene oxide is possible without any problems even if the package is manufactured and/or sealed with the packaging material according to the invention.

According to one embodiment of the invention, the release lacquer according to the invention is a polyamide-based release lacquer and, when the packaging is opened, on the one hand ensures problem-free detachment from the packaging base material and, on the other hand, also ensures sufficient adhesion between the release lacquer and the sealing medium.

According to the invention, the sealing medium is selected from a group which seals against polyolefin(s), in particular polyethylene or polypropylene, and/or against polyesters, in particular polyethylene terephthalate, and/or against paper and/or against polystyrene or mixtures of the aforementioned substances and comprises polyolefin(s), in particular polyethylene or polypropylene, polyester, in particular polyethylene terephthalate, or mixtures of the aforementioned substances, and ethylene-vinyl acetate copolymer. These sealing media are common and inexpensive heat-sealable materials that can be sealed against various substrates, which are made, for example, of polyethylene, polypropylene, polyethylene terephthalate, paper or polystyrene or have sealing surfaces with or made of these materials. In advantageous manner optimum sealing seam strengths may be achievd with the above-mentioned sealing materials, which are in the range from 1.0 N/15 mm to 2.0 N/15 mm.

According to one embodiment of the invention, the sealing medium is applied to the release liner in the form of a dispersion consisting of a mixture of water and ethylene-vinyl acetate copolymer and, optionally, an amount of an alcohol, in particular ethanol, propanol, isopropanol or butanol, in the range of 0.01 wt-% (weight in %) to 8 wt-%, preferably in the range from 0.1 wt-% to 6.5 wt-% and particularly preferably in the range from 3.5 wt-% to 5 wt-%, and/or optionally a further sealing medium.

According to an alternative according to the invention, the dispersion does not contain any solvent, in particular no other organic solvent, apart from water and ethylene-vinyl acetate copolymer.

According to a preferred embodiment of the invention, this dispersion is a heat-sealable dispersion.

According to the invention, the sealing medium is applied to the release coating with an application weight in the range of 0.5 g/m² to 6 g/m², preferably in the range of 1.5 g/m² to 5 g/m² and particularly preferably in the range of 2 g/m² to 4 g/m². Such a coating weight enables the production of a light packaging material and thus the production of a light packaging. At the same time, such a coating weight and the associated quantity of sealing medium ensures an optimal sealing with the respective counterpart of the packaging material according to the invention, both with regard to a tightness of the packaging and with regard to a sealing strength of the sealed seam already in hot condition of the sealed seam and thus an optimal hot-tack.

As mentioned above, the packaging material according to the invention is gas permeable, so that ethylene oxide sterilization of the product contained in a package manufactured and/or sealed with the packaging material is possible without any problems. The gas permeability of the packaging material according to the invention is essentially ensured by the fact that the packaging base material of the packaging material according to the invention consists of paper and/or has at least one paper layer facing the release lacquer and consisting of fibres whose braiding is gas permeable. According to the invention the paper material used for the production of the packaging material is plasma-treated, whereby a fraying of the paper is advantageously avoided.

According to the invention, the release lacquer is applied on the paper layer in a gas-permeable layer thickness.

Furthermore, the packaging material in accordance with the invention is a medical paper.

Furthermore, the objective according to the invention is also solved by a method for producing a packaging material according to the above, wherein the method comprises the following steps:
providing a packaging base material consisting of paper or having an outer paper surface;
coating, in particular over the entire surface, the outer paper surface with a release lacquer; and
applying, at least in sections, a sealing medium on the release lacquer preferably in the form of a grid.

In addition, the objective according to the invention is also solved by using a packaging material with the above properties to produce a container, in particular a medical container.

### SUMMARY

In summary, the subject matter of the invention can thus be described as follows:

The packaging material according to the invention is applicable in the field of and/or as “medical paper” and fulfils all requirements for a “medical paper”. The packaging material according to the invention comprises a sterilizable and heat-sealable coating on paper and is sealable against various substrates, such as predominantly polyethylene, but also against polypropylene and/or polyethylene terephthalate as well as against paper and polystyrene and, if desired, also against mixtures of the aforementioned polyolefins and/or other materials. The packaging material in accordance with the invention allows a clean peel without paper fibre tearing, whereby the coating of the packaging base material is completely transferred to the opposite side, i.e. the counterpart to be sealed, so that the packaging cannot be closed again or an opening of the packaging is directly visible. It is not possible to reseal a packaging once closed with the packaging material according to the invention and then opened. The seal strength achievable with the packaging material according to the invention is in the range from 1.0 N/15 mm to 2.0 N/15 mm.

Since the dispersion used in accordance with the invention, in particular heat-seal dispersion, does not contain any solvent, in particular no other or further organic solvent, except possibly alcohol, the packaging material used in accordance with the invention excludes the presence of such other organic solvent residue as was to be feared in the state of the art.

In an advantageous way, the coating of the packaging material according to the invention, consisting of release lacquer and/or sealing medium, can be applied to the packaging base material by means of gravure printing, which can be carried out in an advantageous way not only very quickly at a high machine speed, but also very precisely and with the creation of a patterned application.

The packaging material according to the invention thus comprises an application of a polyamide-based release lacquer, in particular over the entire surface, which does not prevent ethylene oxide sterilization and ensures clean removal of the heat-seal coating from a respective counterpart.

The heat seal coating is characterized by having a good hot-tack and a wide processing window on a packaging machine and is sealable against a variety of substrates such as polyethylene, polypropylene, polyethylene terephthalate, polystyrene or paper. In addition, the use of a dispersion means that the risk of an increased residual solvent content is kept extremely low or, depending on the dispersion used, avoided altogether. The heat-sealable dispersion is preferably applied as a grid, which ensures that a package manufactured with the packaging material according to the invention can be sterilized with ethylene oxide.

The combination of release coating applied over the entire surface and heat-sealing dispersion applied in a grid simultaneously offers a clean transfer of release coating and sealing medium from the paper without having to fear a fibre tear of the paper to the opposing web. Furthermore, the release coating applied over the entire surface and the heat-seal dispersion applied with a grid pattern ensure sufficient adhesion between release coating and heat-seal dispersion as well as sufficient optimum cohesive force in the release coating to achieve the required peeling force.

Due to the particularly good sealing properties of the heat-seal dispersion, a significantly lower application weight can be chosen. This in turn allows optimum, increased efficiency to be achieved on the coating line.

Further embodiments of the invention result from the dependent claims.

### BRIEF DESCRIPTION OF THE DRAWINGS

In the following, the invention is described with exemplary embodiments on the basis of figures, showing:

FIG. 1 a schematic representation of an embodiment of the invention; and

FIG. 2 a schematic representation of the embodiment of the invention according to FIG. 1, whereby the packaging material according to the invention is first connected to a counterpart and then separated from it again.

### DETAILED DESCRIPTION

In the following, the same reference numbers are used for identical and similarly parts.

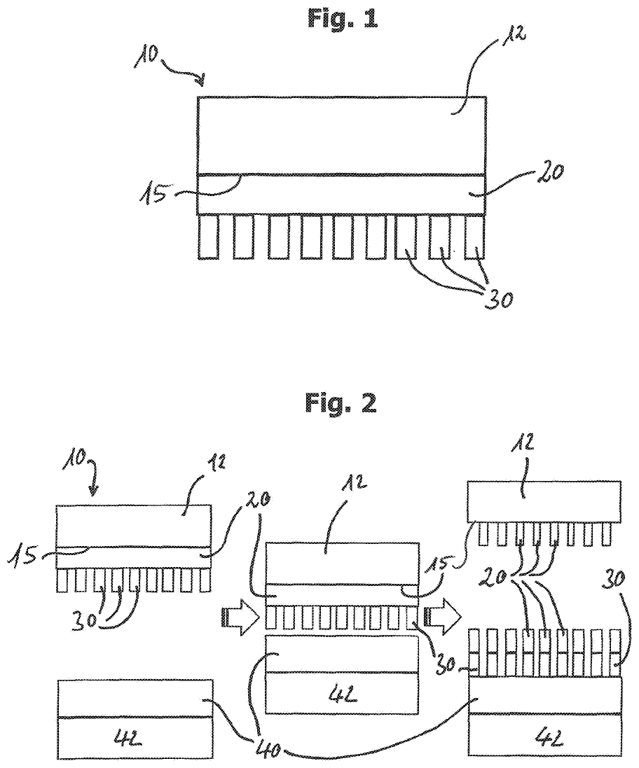

FIG. 1 shows an embodiment of a packaging material 10 according to the invention, which consists of a packaging base material 12 of plasma-treated paper with a basis weight of 60 g/m², on the lower surface of which, as shown in FIG. 1, a release lacquer 20 based on polyamide is applied over the entire surface with an application quantity of 1.5 g/m². A sealing medium 30 is in turn applied to this release lacquer 20 in a grid pattern, the application weight of the sealing medium being 3 g/m².

FIG. 2 shows a schematic representation of the embodiment of the invention according to FIG. 1, wherein the packaging material 10 according to the invention is first connected to a counterpart 40 and then separated from it again. For this purpose, the packaging material 10 according to the invention is firstly joined to the counterpart 40 in such a way that the sealing medium 30, which is connected to the packaging base material 12 via the release lacquer 20, is fed to the counterpart 40, in this case polyethylene, and sealed with it. The counterpart 40 in turn is arranged on a carrier material 42. According to the invention, ethylene-vinyl acetate copolymer is used as sealing medium 30, which seals against polyethylene with an optimal sealing force. When separating the packaging material 10 according to the invention from the counterpart 40, the sealing medium 30 remains on this counterpart 40. Furthermore, with the sealing medium 30, a part of the release lacquer 20 remains at the counterpart 40, wherein the release lacquer 20 breaks during the separation of the packaging material 10 according to the invention from the counterpart 40 and remains at the packaging base material 12 in those areas in which, with an intact packaging material 10, no sealing medium 30 was arranged on the release lacquer 20 before the packaging material 10 was joined to the counterpart 40. Thus, if an attempt were made to rebond the remaining packaging base material 12 with the release coating, a layer also consisting of release coating would be located opposite the counterpart 40, so that a rebonding of the packaging base material 12 with the counterpart 40 is not possible.

According to another example of the invention, a plasma-treated paper web 12 with a basis weight of 80 g/m2 is fully coated with a polyamide-based release lacquer 20. Release lacquer 20 is applied with a basis weight of 1.6 g/m2. In turn, an aqueous heat-sealing dispersion 30 with a basis weight of 3.1 g/m2 is applied to this release coating 20 in a grid pattern.

It should be noted at this point that all the parts described above, taken individually and in any combination, in particular the details shown in the figures, are claimed to be inventive. Modifications of this are familiar to the skilled person.

### REFERENCE SIGNS

10 packaging material
12 packaging base material
15 outer paper surface
20 release lacquer
30 sealing medium
40 counterpart
42 carrier material

1-11. (canceled)

12. A packaging material, in particular for medical purposes, the packaging material comprising:
a packaging base material having an outer paper surface;
a release lacquer that coats an entirety of the outer paper surface; and
a sealing medium that is applied on the release lacquer in a grid pattern.

13. The packaging material according to claim 12, wherein the release lacquer is a polyamide-based release lacquer.

14. The packaging material according to claim 12, wherein the sealing medium comprises polyolefins, or polyester, or a mixture of polyolefins and polyester, and ethylene-vinyl acetate copolymer.

15. The packaging material according to claim 12, wherein the sealing medium is applied on the release lacquer as a dispersion.

16. The packaging material according to claim 15, wherein the dispersion comprises a mixture of water and ethylene-vinyl acetate copolymer.

17. The packaging material according to claim 15, wherein the dispersion further comprises water as a solvent, and is a heat-sealable dispersion.

18. The packaging material according to claim 12, wherein the sealing medium is applied on the release lacquer with an application weight in the range from 0.5 g/m² to 6 g/m².

19. The packaging material according to claim 12, wherein the packaging material is gas permeable.

20. The packaging material according to claim 12, wherein the packaging material is a medical paper.

21. A method for producing a packaging material, comprising:
coating an entirety of an outer paper surface of a packaging base material with a release lacquer; and
applying a sealing medium on the release lacquer in a grid pattern.

22. A method for producing a medical container, comprising:
feeding a sealing medium of a packaging material according to claim 1 to a counterpart material; and
heat-sealing the sealing medium with the counterpart material.

23. The method of claim 22, wherein the sealing medium is ethylene-vinyl acetate copolymer and the counterpart material is polyethylene.

24. The packaging material according to claim 14, wherein the sealing medium comprises polyethylene or polypropylene, or polyethylene terephthalate, or a mixture of polyethylene or polypropylene and polyethylene terephthalate, and ethylene-vinyl acetate copolymer.

25. The packaging material according to claim 14, wherein the sealing medium seals against polyolefins, or polyester, or paper, or polystyrene, or a mixture of at least two of polyolefins, polyester, paper, or polystyrene.

26. The packaging material according to claim 25, wherein the sealing medium seals against polyethylene or polypropylene, or polyethylene terephthalate, or paper, or polystyrene, or a mixture of at least two of polyethylene or polypropylene, polyethylene terephthalate, paper, or polystyrene.

27. The packaging material according to claim 16, wherein the dispersion further comprises an amount of an alcohol selected from the group consisting of ethanol, propanol, isopropanol, or butanol, in the range from 0.01 wt-% to 8 wt-%.

28. The packaging material according to claim 27, wherein the amount of alcohol is in the range from 0.1 wt-% to 6.5 wt-%.

29. The packaging material according to claim 27, wherein the amount of alcohol is in the range from 3.5 wt-% to 5 wt-%.

30. The packaging material according to claim 16, wherein the dispersion further comprises a second sealing medium.

31. The packaging material according to claim 17, wherein the dispersion does not comprise any solvent other than the water.

32. The packaging material according to claim 18, wherein the sealing medium is applied on the release lacquer with an application weight in the range from 1.5 g/m² to 5 g/m².

33. The packaging material according to claim 18, wherein the sealing medium is applied on the release lacquer with an application weight in the range from 2 g/m² to 4 g/m².

34. The packaging material according to claim 12, wherein the packaging base material comprises plasma-treated paper.
